(19) United States
(12) Patent Application Publication
(10) Pub. No.: US 2025/0170089 A1
(43) Pub. Date: May 29, 2025

(54) ANTIMICROBIAL AMINO ESTERS

(71) Applicant: THE HONG KONG POLYTECHNIC UNIVERSITY, Hong Kong (CN)

(72) Inventors: Xiaoming TAO, Hong Kong (CN); Jun LI, Hong Kong (CN); Linlin MA, Hong Kong (CN)

(21) Appl. No.: 18/520,667

(22) Filed: Nov. 28, 2023

(51) Int. Cl. A61K 31/223; A01N 31/14; A01N 37/44; A01N 47/30; A01N 59/00; A01P 1/00; A61K 45/06; A61P 31/04

(52) U.S. Cl. CPC A61K 31/223; A01N 31/14; A01N 37/44; A01N 47/30; A01N 59/00; A01P 1/00; A61K 45/06; A61P 31/04

(57) ABSTRACT

A method of treating an object contaminated with or suspected of being contaminated with one or more microorganisms, the method comprising contacting the object with a compound such that the compound kills or inhibits the growth of at least a portion of the one or more microorganisms, wherein the compound has Formula 1:

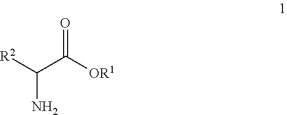

or a pharmaceutically acceptable salt thereof, wherein:
R¹ is C₄-C₂₀ alkyl or C₄-C₂₀ alkene; and
R² is hydrogen, C₁-C₄ alkyl, or a side chain of a naturally occurring amino acid.

### TECHNICAL FIELD

The present disclosure generally relates to the use of amino esters as antimicrobial agents.

### BACKGROUND

Microbial infections, which can lead to many diseases or even death, have always been an important issue for public health worldwide. Developing antimicrobial materials or agents are thus of significant importance to reduce the morbidities and mortalities caused by microbial infections. To date, several kinds of antimicrobial materials have been reported. The first category is metal-based antimicrobial materials, which includes silver, copper, zinc and other metal ions, oxides and chalcogenides. However, the use of heavy metals may raise safety concerns. Another area of development is antimicrobial peptides (AMPs), which typically are typically peptide oligomers comprising dozens of amino acids. AMPs generally show very high antimicrobial activities, However, their stability against pH and thermal change is not sufficient due to their protein-like structure. Cationic polymers with guanidine and quaternary ammonium end groups have shown promising applications in antimicrobial surface treatment, but their biocompatibility is generally poor. Some natural products, typically essential oil (EOs) and chitin, have also been reported to exhibit antimicrobial properties.

### SUMMARY

Provided herein is a series of wide-spectrum antimicrobial amino esters with very clear chemical structures and the structure-activity relationships. The amino esters can be prepared from lactic acid, amino acids and fatty alcohols through a simple esterification reaction and purified by recrystallization. Apart from the excellent antimicrobial properties, the amino esters are biodegradable, biocompatible, cost-effective and easy to be batch produced. The relationship between the chemical structure and antimicrobial efficacy has also been investigated and this may provide a general strategy to design green antimicrobial materials.

In a first aspect, provided herein is a method of treating an object contaminated with or suspected of being contaminated with one or more microorganisms, the method comprising contacting the object with a compound such that the compound kills or inhibits the growth of at least a portion of the one or more microorganisms, wherein the compound has Formula 1:

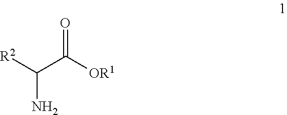

or a pharmaceutically acceptable salt or a zwitterion thereof, wherein:
R¹ is C₄-C₂₀ alkyl or C₄-C₂₀ alkene; and
R² is hydrogen, C₁-C₄ alkyl, or a side chain of a naturally occurring amino acid.

In certain embodiments, R¹ is C₁₀-C₂₀ alkyl or C₁₀-C₂₀ alkene.

In certain embodiments, R¹ is C₁₀-C₁₈ alkyl or C₁₀-C₁₈ alkene.

In certain embodiments, R² is selected from the group consisting of:

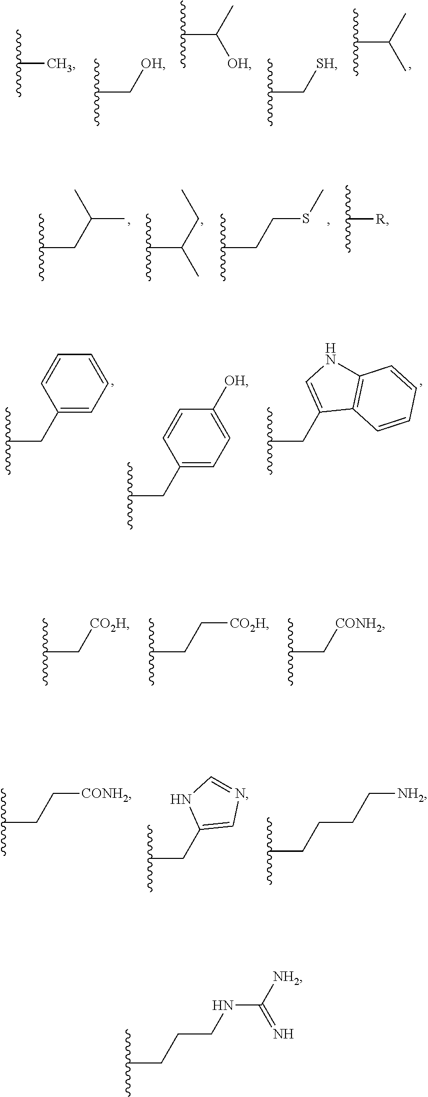

and pharmaceutically acceptable salt or zwitterions thereof, wherein R and N together with the carbon to which they are attached form a 5 membered heterocycloalkyl.

In certain embodiments. R² is hydrogen or C₁-C₂ alkyl.

In certain embodiments, R¹ is C₁₀-C₁₈ alkyl or C₁₀-C₁₈ alkene; and R² is hydrogen or C₁-C₂ alkyl.

In certain embodiments. R¹ is C₁₀-C₁₈ alkyl and R² is C₁-C₂ alkyl

In certain embodiments, R¹ is C₁₂ alkyl and R² is methyl.

In certain embodiments, the one or microorganisms comprise a bacterium, a fungus, a virus, or a mixture thereof.

In certain embodiments, the one or microorganisms comprise a gram-positive bacterium or a gram-negative bacterium.

In certain embodiments, the one or microorganisms are selected from the group consisting of E. coli, S. aureus, C. albicans, and methicillin-resistant S. aureus,

In certain embodiments, the method comprises contacting the object with a disinfectant composition comprising the compound and a solvent.

In certain embodiments, the solvent comprises water.

In certain embodiments, the compound is present in the solvent in the form of nanoparticles.

In certain embodiments, the nanoparticles have an average diameter of 50-200 nm.

In certain embodiments, the compound is present in the disinfectant composition at a concentration of at least 80 μg/mL.

In certain embodiments, the compound is present in the disinfectant composition at a concentration of 90 to 500 μg/mL.

In certain embodiments, the disinfectant composition further comprises a quaternary ammonium chloride, triclosan, or triclocarban.

### BRIEF DESCRIPTION OF THE DRAWINGS

The foregoing aspects and many of the attendant advantages of this invention will become more readily appreciated by reference to the following detailed description, when taken in conjunction with the accompanying drawings.

FIG. 1 depicts a 1H NMR spectrum of Ala4 in CDCl3.

FIG. 2 depicts a high-resolution mass spectrum of Ala4.

FIG. 3 depicts a 1H NMR spectrum of Ala12 in CD₃OD.

FIG. 4 depicts a high-resolution mass spectrum of Ala12.

FIG. 5 depicts a dynamic light scatting (DLS) spectrum of Ala12.

FIG. 6 depicts the results of antimicrobial testing of Ala12 in E. coli, S. aureus, C. albicans, and methicillin-resistant S. aureus (MRSA) in accordance with certain embodiments described herein.

FIG. 7 depicts a table presenting the minimum inhibitory concentration of (MIC) of different esters against E. coli and S. aureus.

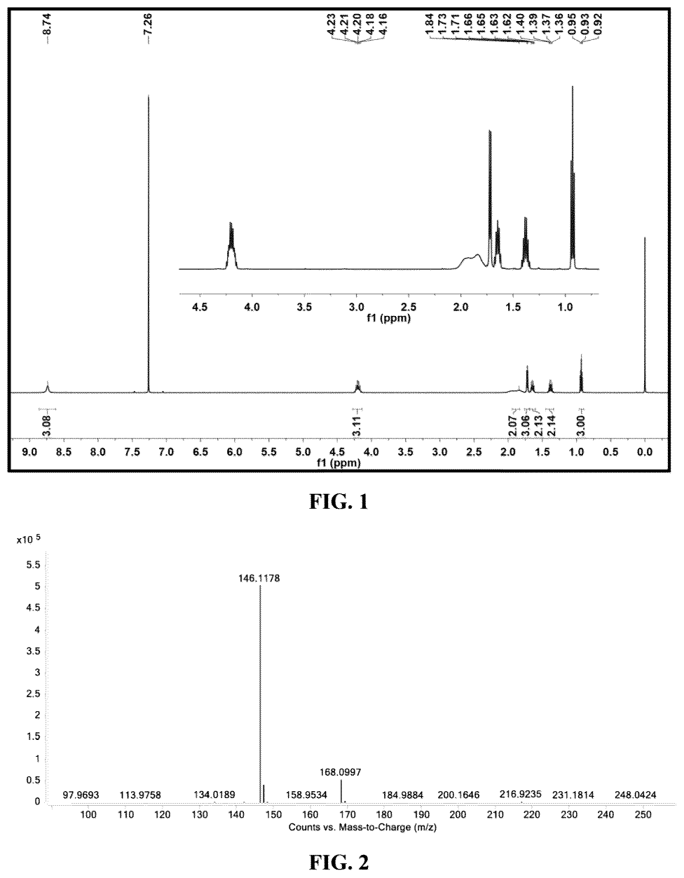

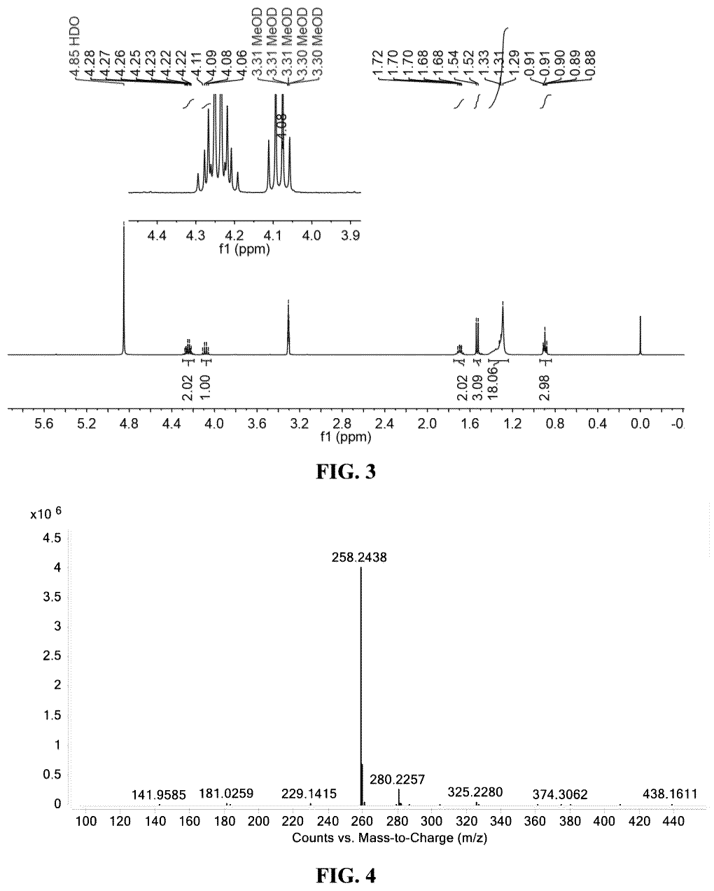

### DETAILED DESCRIPTION

### Definitions

Throughout the present disclosure, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present disclosure and claims, unless the context requires otherwise, the word “include” or variations such as “includes” or “including”, will be understood to imply the inclusion of a stated integer or group of integers, but not the exclusion of any other integer or group of integers.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±7%, ±5%, ±3%, ±1%, or ±0% variation from the nominal value unless otherwise indicated or inferred.

The term “Gram-positive bacteria” as used herein refers to bacteria characterized by having as part of their cell wall structure peptidoglycans as well as polysaccharides and/or teichoic acids and are characterized by their blue-violet color reaction in the Gram-staining procedure.

The term “Gram-negative bacteria” as used herein refers to bacteria characterized by the presence of a double membrane surrounding each bacterial cell and are characterized by the absence of color upon washing out with a decolorizer and counter-staining pink with safranin in the Gram-staining procedure.

The term “composition” is intended to encompass a product comprising the specified ingredients in the specified amounts, as well as any product that results, directly or indirectly, from combinations of the specified ingredients in the specified amounts.

As used herein, unless otherwise indicated, the phrase “pharmaceutically acceptable salt(s)” includes salts of acidic or basic groups which may be present in the compounds described herein. The compounds described herein that contain basic groups, such as amines, are capable of forming a wide variety of salts with various inorganic and organic acids. The acids that may be used to prepare pharmaceutically acceptable acid addition salts of such basic compounds described herein are those that form relatively non-toxic acid addition salts, i.e., salts containing pharmacologically acceptable anions, such as acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phosphate/diphosphate, polygalacturonate, salicylate, stearate, subacetate, succinate, tannate, tartrate, teoclate, tosylate, triethiodode, and valerate salts.

In other cases, the compounds described herein may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Provided herein is a method of treating an object contaminated with or suspected of being contaminated with one or more microorganisms, the method comprising contacting the object with a compound such that the compound kills or inhibits the growth of at least a portion of the one or more microorganisms, wherein the compound has Formula 1:

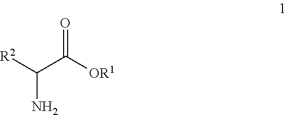

or a pharmaceutically acceptable salt or a zwitterion thereof, wherein:
R¹ is C₄-C₂₀ alkyl or C₄-C₂₀ alkene; and
R² is hydrogen, C₁-C₄ alkyl, or a side chain of a naturally occurring amino acid.

R¹ can be a liner chain alkyl, liner chain alkenyl, branched chain alkyl, branched chain alkenyl, cycloalkyl, or cycloalkenyl. In certain embodiments, R¹ is C₅-C₂₀ alkyl, C₆-C₂₀ alkyl, C₇-C₂₀ alkyl, C₈-C₂₀ alkyl, C₉-C₂₀ alkyl, C₁₀-C₂₀ alkyl, C₁₁-C₂₀ alkyl, C₁₂-C₂₀ alkyl, C₁₂-C₁₉ alkyl, C₁₂-C₁₈ alkyl, C₁₂-C₁₇ alkyl, C₁₂-C₁₆ alkyl, C₁₂-C₁₅ alkyl, C₁₂-C₁₄ alkyl, C₁₂-C₁₃ alkyl, C₈-C₁₆ alkyl, C₉-C₁₅ alkyl, C₁₀-C₁₄ alkyl, C₁₁-C₁₃ alkyl, R¹ is C₅-C₂₀ alkenyl, C₆-C₂₀ alkenyl, C₇-C₂₀ alkenyl, C₈-C₂₀ alkenyl, C₉-C₂₀ alkenyl, C₁₀-C₂₀ alkenyl, C₁₁-C₂₀ alkenyl, C₁₂-C₂₀ alkenyl, C₁₂-C₁₉ alkenyl, C₁₂-C₁₈ alkenyl, C₁₂-C₁₇ alkenyl, C₁₂-C₁₆ alkenyl, C₁₂-C₁₅ alkenyl, C₁₂-C₁₄ alkenyl, C₁₂-C₁₃ alkenyl, C₈-C₁₆ alkenyl, C₉-C₁₅ alkenyl, C₁₀-C₁₄ alkenyl, or C₁-C₁₃ alkenyl. In instances in which R¹ is C₄-C₂₀ alkene, R¹ can comprise 1, 2, 3, or olefins. Exemplary R¹ groups include, but are not limited to, n-decanyl, n-undecanyl, n-dodecanyl, n-tridecanyl, n-tetradecanyl, n-pentadecanyl, and n-hexadecanyl. In certain embodiments, R¹ is n-dodecanyl.

R² can be a liner chain alkyl, branched chain alkyl, cycloalkyl, or hydrogen. In certain embodiments, R² is hydrogen, C₁-C₄ alkyl, C₁-C₃ alkyl, or C₁-C₂ alkyl. Exemplary R² groups include hydrogen, methyl, ethyl, n-propyl, isopropyl, n-butyl, isobutyl, and t-butyl. In certain embodiments, R² is methyl.

In instances in which R² is a side chain of a naturally occurring amino acid, R² can be selected from the group consisting of R² is selected from the group consisting of:

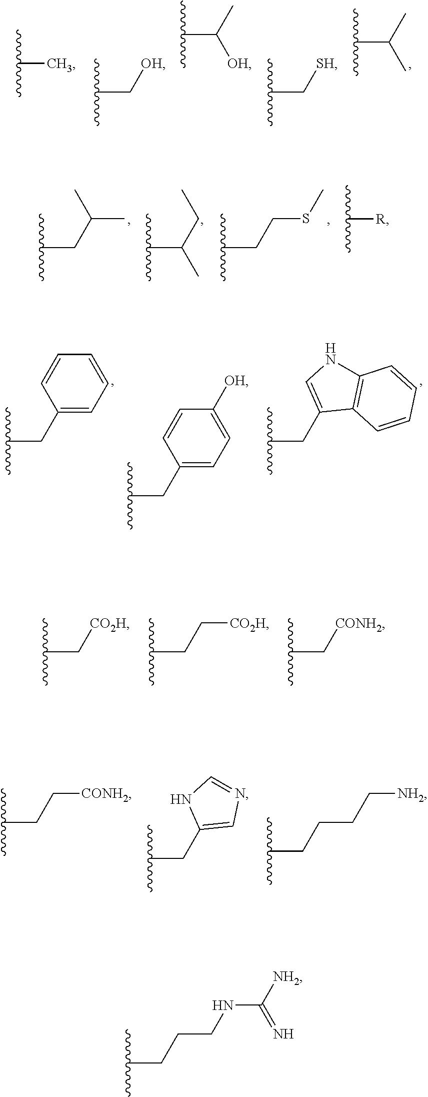

and pharmaceutically acceptable salt or zwitterion thereof, wherein R and N together with the carbon to which they are attached form a 5 membered heterocycloalkyl (i.e., proline).

In certain embodiments, the compound of formula 1 has the formula:

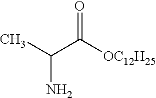

or a pharmaceutically acceptable salt thereof.

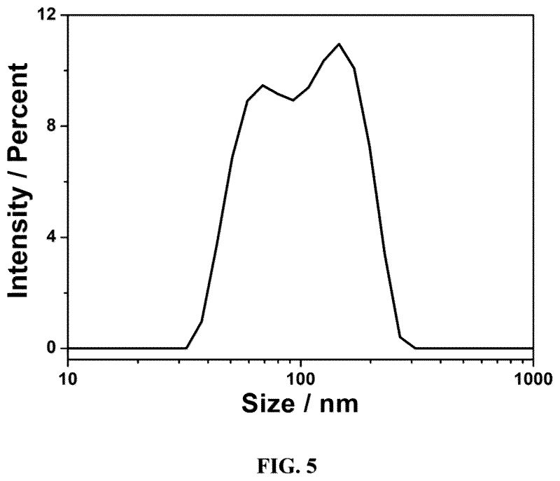

The dynamic light scattering (DLS) and zeta-potential indicate that Ala12 assembles into nanoparticles with an average size of around 50-200 nm (FIG. 5) and the surface potential of the generated nanoparticle can be +2.15 mV, which means the nanoparticle is positive charged and the hydrophilic —NH₂ groups are probably distributed on the outer surfaces. The self-assembly of the positive charged nanoparticles will attack the negative-charged membranes of the microbial through electrostatic attraction, which is more effective than the Brownian movement of the neutral ones and is thus dominant for the high antimicrobial efficacy.

The bacteria can be Gram-positive bacteria, Gram-negative bacteria, Gram-variable bacteria, or Gram-indeterminate bacteria.

Exemplary Gram-negative bacteria include, but are not limited to, Acinetobacter calcoaceticus, Acinetobacter baumannii, Actinobacillus actinomycetemcomitans, Aeromonas hydrophila, Alcaligenes xylosoxidans, Bacteroides, Bacteroides fragilis, Bartonella bacilliformis, Bordetella spp., Borrelia burgdorferi, Branhamella catarrhalis, Brucella spp., Campylobacter spp., Chlamydia pneumoniae, Chlamydia psittaci, Chlamydia trachomatis, Chromobacterium violaceum, Citrobacter spp., Eikenella corrodens, Enterobacter aerogenes, E. coli, Flavobacterium meningosepticum, Fusobacterium spp., Haemophilus influenzae, Haemophilus spp., Helicobacter pylori, Klebsiella pneumoniae, Klebsiella spp., Legionella spp., Leptospira spp., Moraxella catarrhalis, Morganella morganii, Mycoplasma pneumoniae, Neisseria gonorrhoeae, Neisseria meningitidis, Pasteurella multocida, Plesiomonas shigelloides, Prevotella spp., Proteus spp., Providencia rettgeri, Pseudomonas aeruginosa, Pseudomonas spp., Rickettsia prowazekii, Rickettsia rickettsii, Rochalimaea spp., Salmonella spp., Salmonella typhimurium, Serratia marcescens, Shigella spp., Shigella sonnei, Treponema carateum, Treponema pallidum, Treponema pallidum endemicum, Treponema pertenue, Veillonella spp., Vibrio cholerae, Vibrio vulnificus, and Yersinia enterocolitica, Yersinia pestis.

Exemplary Gram-positive bacteria include, but are not limited to, Actinomyces spp., Bacillus anthracis, Bifidobacterium spp., Clostridium botulinum, Clostridium perfringens, Clostridium spp., Clostridium tetani, Corynebacterium diphtheriae, Corynebacterium jeikeium, Enterococcus faecalis, Enterococcus faecium, Erysipelothrix rhusiopathiae, Eubacterium spp., Gardnerella vaginalis, Gemella morbillorum, Leuconostoc spp., Mycobacterium abscessus, Mycobacterium avium complex, Mycobacterium chelonae, Mycobacterium fortuitum, Mycobacterium haemophilium, Mycobacterium kansasii, Mycobacterium leprae, Mycobacterium marinum, Mycobacterium scrofulaceum, Mycobacterium smegmatis, Mycobacterium terrae, Mycobacterium tuberculosis, Mycobacterium ulcerans, Nocardia spp., Peptococcus niger, Peptostreptococcus spp., Proprionibacterium spp., Sarcina lutea, Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae (group B streptococcus), Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes (group A streptococcus), and Streptococcus salivarius, Streptococcus sanguis.

The MIC of the compounds described herein against Escherichia coli (E. coli, ATCC No: 25922) and Staphylococcus aureus (S. aureus, ATCC No: 6538) were measured by the constant broth dilution method, as shown in FIG. 7. It is clear to see that the end group and the alkyl chain length significantly influence the antimicrobial properties and the positive-charged —NH₂ group will significantly reduce the MIC as compared with the neutral —OH group.

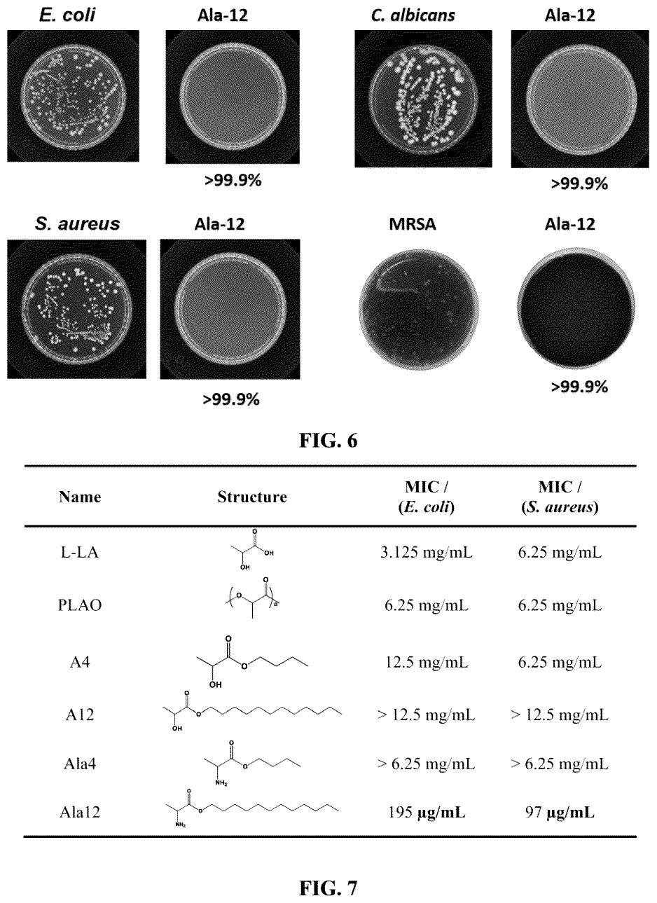

The antimicrobial properties of Ala12 against fungi (Candida albicans), methicillin-resistant Staphylococcus aureus (MRSA) and the MIC were 390 μg/m, respectively. The rapid bactericidal test of Ala12 (standard: ASTM E2149 13a 1015) shows that the amino ester kills microbes within 10 minutes. Ala12 exhibits exceptional biosafety and is completely free of skin irritation and skin sensitization at a concentration as high as 3.9 mg/mL, according to standard ISO 10993-10:2010. The results of antimicrobial testing of Ala14 against E. coli, S. aureus, C. albicans, and MRSA are shown in FIG. 6.

The object can be an animal or a non-animal object. The animal (e.g., a mammal) can include humans, non-human primates, canines, felines, and rodents.

Non-animal objects can include, but are not limited to, surfaces (e.g., of domiciliary and hospitals), curtains, textiles, appliances, food processing equipment, military equipment, personal protective gear, medical devices, domestic objects, and building structures.

The compounds described herein can be prepared using any number of synthetic protocols. The selection of the appropriate synthetic method is well within the skill of a person of ordinary skill in the art. In certain embodiments, the compounds described herein are prepared by condensation of an amino acid as illustrated in the reaction scheme below.

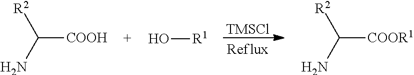

wherein R¹ and R² are as defined herein.

In a typical procedure the amino acid, fatty alcohol and chlorotrimethylsilane (TMSCl), are reacted neat in a molar ratio of 1:4:5 by reflux for 4 hours and then cooled down to room temperature. The amino ester products were recrystallized from cold ether/ethanol solution as white precipitates, and the chemical structures were fully characterized by ¹H NMR and HPLC-MS spectra and matches very well with the predicted spectra (See, e.g., FIGS. 3 and 4).

### EXAMPLES

### Example 1—Synthesis of Ala4

For a typical synthesis of Ala4, 1.00 g Alanine (11.2 mmol) and 3.50 g 1-butanol (44.8 mmol) were firstly added into the single-neck flask, and then the chlorotrimethylsilane (6.05 g, 56.0 mmol) was added into the mixture slowly. Then the mixture was allowed to reflux for 4 hours. The excess butanol and the side products were removed by rotary evaporation. Ala4 was recrystallized from cold ether/ethanol as white powders.

### Example 2—Synthesis of Ala12

For a typical synthesis of Ala4, 1.00 g Alanine (11.2 mmol) and 8.43 g lauric alcohol (44.8 mmol) were firstly added into the single-neck flask, and then the chlorotrimethylsilane (6.05 g, 56.0 mmol) was added into the mixture slowly. Then the mixture was allowed to reflux for 4 hours. The excess lauric alcohol and the side products were removed by rotary evaporation. Ala12 was recrystallized from cold ether/ethanol as white powders.

### Example 3—Antimicrobial Test

The Ala12 was diluted to 10 mg/mL with phosphate buffer. The shaking method (see the ASTM standard E2149 13a 2015) was used to detect their antibacterial activity, wherein the PBS group was used as a blank control. The contact time of bacteria treated with Ala12 is 10 mins.

What is claimed is:

1. A method of treating an object contaminated with or suspected of being contaminated with one or more microorganisms, the method comprising contacting the object with a compound such that the compound kills or inhibits the growth of at least a portion of the one or more microorganisms, wherein the compound has Formula 1: 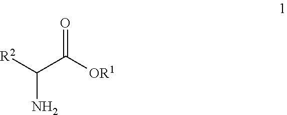
or a pharmaceutically acceptable salt or a zwitterion thereof, wherein:
R¹ is C₄-C₂₀ alkyl or C₄-C₂₀ alkene; and
R² is hydrogen, C₁-C₄ alkyl, or a side chain of a naturally occurring amino acid.

2. The method of claim 1, wherein R¹ is C₁₀-C₂₀ alkyl or C₁₀-C₂₀ alkene.

3. The method of claim 1, wherein R¹ is C₁₀-C₁₈ alkyl or C₁₀-C₁₈ alkene.

4. The method of claim 1, wherein R² is selected from the group consisting of: 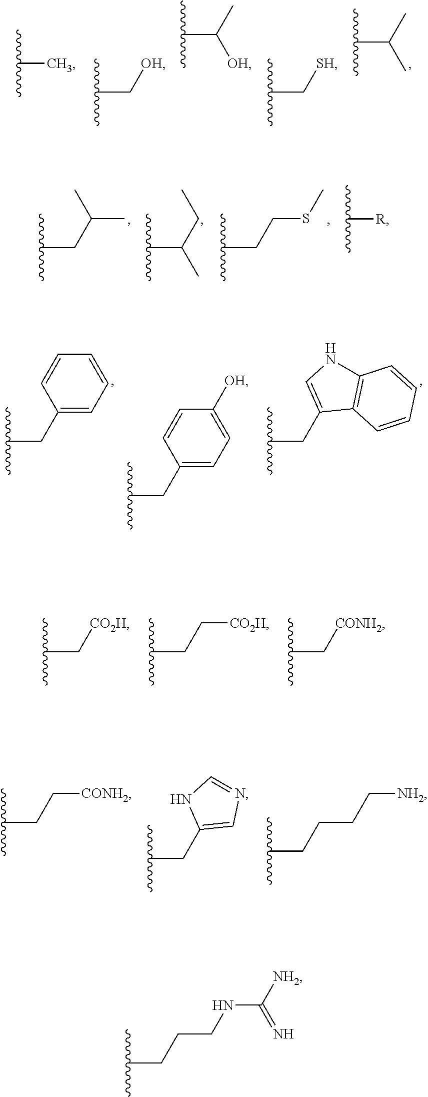
and pharmaceutically acceptable salt or zwitterions thereof, wherein R and N together with the carbon to which they are attached form a 5 membered heterocycloalkyl.

5. The method of claim 1, wherein R² is hydrogen or C₁-C₂ alkyl.

6. The method of claim 1, wherein R¹ is C₁₀-C₁₈ alkyl or C₁₀-C₁₈ alkene; and R² is hydrogen or C₁-C₂ alkyl.

7. The method of claim 1, wherein R¹ is C₁₀-Cis alkyl and R² is C₁-C₂ alkyl

8. The method of claim 1, wherein R¹ is C₁₂ alkyl and R² is methyl.

9. The method of claim 1, wherein the one or microorganisms comprise a bacterium, a fungus, a virus, or a mixture thereof.

10. The method of claim 1, wherein the one or microorganisms comprise a gram-positive bacterium or a gram-negative bacterium.

11. The method of claim 8, wherein the one or microorganisms are selected from the group consisting of E. coli, S. aureus, C. albicans, and methicillin-resistant S. aureus,

12. The method of claim 1, wherein the method comprises contacting the object with a disinfectant composition comprising the compound and a solvent.

13. The method of claim 12, wherein the solvent comprises water.

14. The method of claim 12, wherein the compound is present in the solvent in the form of nanoparticles.

15. The method of claim 14, wherein the nanoparticles have an average diameter of 50-200 nm.

16. The method of claim 12, wherein the compound is present in the disinfectant composition at a concentration of at least 80 μg/mL.

17. The method of claim 12, wherein the compound is present in the disinfectant composition at a concentration of 90 to 500 μg/mL.

18. The method of claim 12, wherein the disinfectant composition further comprises a quaternary ammonium chloride, triclosan, or triclocarban.
